(19) United States
(12) Patent Application Publication
(10) Pub. No.: US 2003/0109028 A1
(43) Pub. Date: Jun. 12, 2003

(54) Method and device for the microbiological decomposition of harmful substances in fluids

(76) Inventor: Alain Constans, Quebec (CA)

Correspondence Address: BIRCH STEWART KOLASCH & BIRCH, PO BOX 747, FALLS CHURCH, VA, 22040-0747, US

(21) Appl. No.: 10/204,848

(22) PCT Filed: Feb. 23, 2001
(86) PCT No.: PCT/EP01/02097

(30) Foreign Application Priority Data
Feb. 25, 2000 (EP) 001040096.6

(51) Int. Cl. C12S 13/00; C12M 1/00

(52) U.S. Cl. 435/262.5; 435/289.1

(57) ABSTRACT

A process for the microbiological degradation of noxious matters in a fluid loaded with noxious matters, wherein an immobilized ensemble of microorganisms (biocoenosis) is employed for microbiological degradation, said biocoenosis being alternately subjected to submerged and non-submerged conditions in a device for the microbiological degradation of noxious matters in fluids, and a device for the microbiological degradation of noxious matters in fluids comprising at least two intake spaces (1, 2) for the fluid, immobilized microorganisms being provided in at least one intake space (1, 2). The device is characterized in that said at least two intake spaces (1, 2) can be brought in mutual fluid connection through a communication duct (3), and that means (4) are provided by which the fluid can be transferred alternately from one intake space (1) to the other intake space, wherein said microorganisms in said at least one intake space (1, 2) can be alternately subjected to submerged and non-submerged conditions.

The present invention relates to a process and device for the microbiological degradation of noxious matters in fluids.

The degradation of noxious matters in fluid systems is often a technically demanding problem. Especially in recent times, methods have become established in which microorganisms are used for degrading noxious substances. For example, biological water-processing plants have become established. However, on the one hand, these are large-scale plants, for example, municipal installations, or else relatively small units employed in the form of home installations. However, it is exceedingly expensive technically to treat problematic fluids in microbiological plants, because low variations in the compositions of the fluids have consequences for the plant involved which are difficult to overlook.

For example, the waste water obtained in restaurants often exhibits substantial variations in composition. For example, fat-containing waste waters are followed by alkaline waste waters when the rinsing machine is being operated. Thus, microorganisms which can accomplish certain tasks as specialists have difficulties to meet their functions reliably under changing conditions.

The state of the art in the treatment of fat-containing waste waters is the use of so-called fat separators, utilizing the physical effect of gravity separation for separating off supernatant fats. Such fat separators are not capable of eliminating fats. The fat remains with the producer and must be disposed of separately. Often, there is an offensive smell when the fat is stored in the fat separator. Through-breaking fat can lead to complications in the waste water system. In addition, conditions imposed by the water authorities require a lower load of fat in the waste water.

GB-A-2 195 624 relates to a waste-water treatment plant in which a fixed biomass and the waste water are disposed in a treatment tank. The biomass is fixed on a support whose surface is normally above the waste water level, and other surfaces which can submerge in the waste water. Anaerobic conditions are thereby provided in the waste water container.

WO-A-98/32703 relates to a device for intensive biological waste-water treatment with freely moveable immersed biofilm, combined with mixing devices and, optionally, the additional introduction of air. The freely moveable immersed biofilm is used to immobilize specific microorganisms in certain areas for waste-water treatment. This device ensures intensification of the treatment process due to the additional freely moveable, suspended/floating immersed biofilm and its proportional oxygen supply from a special mixing device, provided upon emergence into the atmosphere in addition to aeration from fine bubbles. A substantially greater utilization of oxygen is obtained by enriching the air of the recycled sludge or waste-water through multiphase pumps. The device described combines the activated sludge process with the freely moveable immersed biofilm process. Said freely moveable biofilm is introduced into a sewage-treatment basin as a biofilm carrier with an activated surface to immobilize specific microorganisms in association with appropriate mixing devices having a scooping effect due to paddles and the proportion of activated sludge. Waste-water flows through the sewage treatment basin which comprises a retaining device for the freely moveable immersed biofilm.

JP-A-09085273 relates to a perforated rotation cylinder packed with supports which have immobilized bacteria supported thereon. The rotation cylinders are rotated. The rotational speed is made variable to achieve variable conditions in the waste-water treatment container. The device described therein allows for the miniaturization of corresponding waste-water treatment plants.

The object of the invention is to provide a process and a device by which microorganisms can be successfully employed for degrading noxious matters in a universal way, if possible, while avoiding the drawbacks encountered in the prior art.

The object of the invention is achieved by a process according to claim 1, and a device according to claim 8.

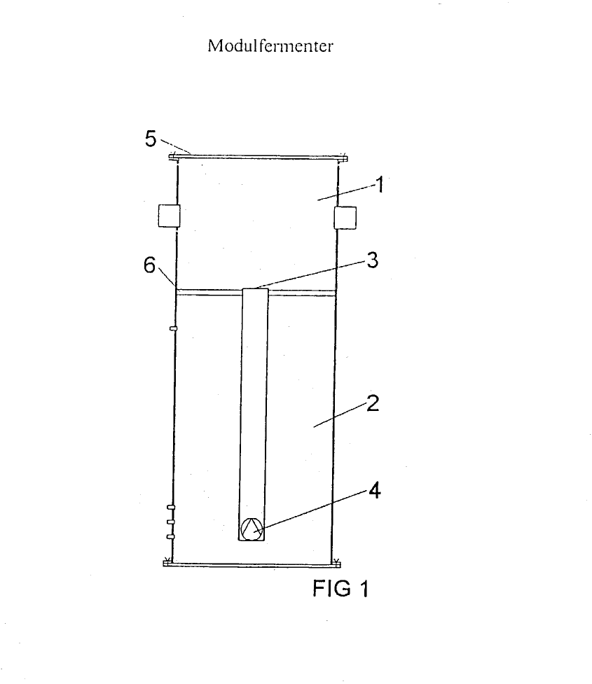

FIG. 1 shows a device for performing the process according to the invention.

The process according to the invention for the microbiological degradation of noxious matters in a fluid loaded with noxious matters employs an immobilized ensemble of microorganisms (biocoenosis) for microbiological degradation, said biocoenosis being alternately subjected to submerged and non-submerged conditions.

According to the invention, the fluid may be a waste water or gas stream or the result of passing a gas stream through a solution.

According to the invention, there may be additionally effected changes of temperature, oxygen content, pH value, osmosis and load by varying the volume and load of the fluid, or combinations thereof.

Preconditioned microorganisms are preferably employed.

The process according to the invention advantageously ensures the alternate provision of substantially anaerobic or substantially aerobic conditions by the change between submerged and non-submerged conditions.

The time intervals for which the microorganisms are subjected to submerged conditions and the time intervals for which the microorganisms are subjected to non-submerged conditions may have different lengths. The time intervals for which the microorganisms are subjected to submerged conditions and the time intervals for which the microorganisms are subjected to non-submerged conditions may have, among themselves respectively, equal or different lengths or be varied in time depending on their number.

The device according to the invention as illustrated in FIG. 1 is a device for the microbiological degradation of noxious matters in fluids comprising:

at least two intake spaces for the fluid, immobilized microorganisms being provided in at least one of said intake spaces;

characterized in that

said at least two intake spaces can be brought in mutual fluid connection through a communication duct, and that means are provided by which the fluid can be transferred alternately from one intake space to the other intake space, wherein said microorganisms in said at least one intake space can be alternately subjected to submerged and non-submerged conditions.

Preferably, the intake spaces are provided within a container and separated from each other by at least one dividing wall.

In a preferred embodiment, the device according to the invention has a pressing and/or sucking pump for pumping over the fluid between the intake spaces.

According to the invention, the pump changes the hydrostatic pressure prevailing in one of the intake spaces for pumping over the fluid against gravity into the other intake space provided above the one intake space, and from the other intake space back into the one intake space.

In a particular embodiment of the device according to the invention, several groups of at least two intake spaces are in fluid connection in parallel and/or in series. Preferably, a means for transferring the fluid from at least one of the intake spaces of one group into at least one of the intake spaces of a neighboring group is provided between said groups of at least two intake spaces.

The module fermenter is particularly suitable for the biological degradation of lipophilic substances from waste waters, especially household effluents, such as kitchen effluents. In principle, however, other waste waters which contain biodegradable substances can also be processed. The invention will be explained in an exemplary manner for the treatment of fat-containing effluents from commercial kitchens.

Previous experiments with biologically working waste-water treatment plants failed because of the sensitivity of the microorganisms towards highly variable introducing conditions.

On the basis of this fact, a system has been developed which takes account of these conditions.

The module fermenter essentially consists of an integrator stage and a fermenter stage.

Integrator Stage:

By an appropriate physical pretreatment, coarse components of >1.5 mm are withdrawn from the kitchen effluent obtained. The thus pretreated waste water is introduced to the integration stage discontinuously depending on the amount obtained. One or more containers which are usually cylindrical in shape form the integration stage. When several containers are employed, the connections are predominantly selected to provide a system of communicating vessels.

Kitchen effluents are highly variable in terms of volume flow rate, temperature, pH value, pollutant load and content of surfactants. In particular periods of time, the supply of waste water is completely cut off (weekend, holidays).

In the integrator stage, the discontinuous stream of waste water is collected. The controlled mixing of several lots of waste water over the day causes a diluting or buffering effect which results in a flattening of the load peaks of the mentioned parameters. The filling height of the integrators is influenced by means of an adaptive control in such a way that as continuous as possible an effluent stream with low variations of the disturbing parameters is released to the fermenter stage.

The integrator stage is designed in such a way as to mimic a static fat separator in connection with the downstream fermenter stage in case the transport of the waste water through the fermenter stage should be disturbed (operation in emergency mode). In the containers of the integrator stage, “anaerobic biology” and/or aerobic biology can prevail, incubated by naturally occurring organisms or by the deliberately induced incubation from outside with preselected microorganisms or biocoenoses.

Fermenter Stage

Several so-called fermenter lines form the fermenter stage. Fermenter lines are assembled from usually two fermenters in series or one pair of fermenters each connected in parallel in such a form that the respectively first fermenter (or the first pair of fermenters) is incubated with a biocoenosis A which is optimized, for example, for the degradation of lipophilic substances, while the second fermenter (or the second pair of fermenters) is incubated with a biocoenosis B which is optimized, for example, for the degradation of COD.

Fermenter

A fermenter typically consists of two separated reaction spaces. An external jacket enclosed, for example, a cylindrical space which is itself divided into upper and lower reaction spaces, preferably by means of a dividing plate. The reaction spaces are interconnected through an ascending pipe which is attached to the dividing plate and creates a communication between the upper reaction space and the lower reaction space. The ascending pipe extends into the lower reaction space down to a few centimeters above the ground. The fermenter container has several receptor pieces for measuring sensors. Further, one inlet and one outlet piece are provided in the upper reaction space. The effective draining level of the outlet piece can be varied by engaging pieces of tubing over it. Thus, a system of successive fermenters can be assembled, wherein the effective draining level of the outlet piece in the subsequent fermenter is respectively lower than that of the previous.

In the lower reaction space, there are connection pieces for measuring sensors and for compressed air and for releasing the lower reaction space.

By appropriately pressurizing the lower reaction space with compressed air, any liquid medium contained therein is forced through the ascending pipe into the upper reaction space. When the pressure in the lower reaction space is released, the medium will flow back into it.

An adaptive control influences the pressurizing with compressed air in such a way that a commuting operation is possible which by the occasional rising of the liquid level above a certain level initiates the discharge of liquid into the respectively downstream fermenter or the downstream pair of fermenters or, in the case of the last fermenter, into the canalization.

Biofilm

To increase the effective surface area, the reaction spaces are preferably provided with a support, especially a bionet. Such a bionet consists, for example, of a plastic weave as the support material and, in particular, a coating which facilitates colonization with microorganisms, such as an agar coating, which by the well-aimed addition of particular substances can be conditioned in such a way that it provides an ecological niche for the desired biocoenosis which provides it with a growth advantage over microorganisms which naturally occur in the waste water. In particular, the preselected microorganisms are immobilized to the support material in a process which takes several days.

Surprisingly, an enrichment with fat or intermediates of fat degradation occurs in the region of the biofilm. This enrichment leads to an improvement of the resistance of the biocoenosis against times of deficiency during which the depots formed are exploited.

Due to the rising and lowering of the liquid level in the fermenter, aerobic and anaerobic conditions alternately prevail in the reaction spaces in a way similar to the effect of low and high tide on the tide line of the sea shores. These perpetually changing conditions result in stress being imposed on the microorganisms. It is found that this stress induces the microorganisms to dissimilate available food sources in the first place rather than accumulate them in the form of biomass, which would ultimately lead to sludge formation in the fermenter and discharge of microorganisms into the canalization.

Control

The mode of operation described above requires the continuous monitoring of relevant parameters and the consideration of static data, such as holiday times, weekends etc. Thus, before weekends, the degree of filling in the integrators and/or fermenters will be chosen particularly high to ensure stock-piling to some extent. When these data change, the controlling program of the module fermenter can be updated or changed on-line.

1. A process for the microbiological degradation of noxious matters in a fluid loaded with noxious matters, wherein an immobilized ensemble of microorganisms (biocoenosis) is employed for microbiological degradation, said biocoenosis being alternately subjected to submerged and non-submerged conditions in a device for the microbiological degradation of noxious matters in fluids.

2. The process according to claim 1, wherein said fluid is a waste water or gas stream or the result of passing a gas stream through a solution.

3. The process according to either of claims 1 or 2, wherein there are additionally effected changes of temperature, oxygen content, pH value, osmosis and load by varying the volume and load of the fluid, or combinations thereof.

4. The process according to at least one of claims 1 to 3, wherein said microorganisms have been preconditioned.

5. The process according to at least one of claims 1 to 4, wherein substantially anaerobic or substantially aerobic conditions are alternately provided by the change between submerged and non-submerged conditions.

6. The process according to any of claims 1 to 5, characterized in that the time intervals for which the microorganisms are subjected to submerged conditions and the time intervals for which the microorganisms are subjected to non-submerged conditions have different lengths.

7. The process according to claim 6, characterized in that the time intervals for which the microorganisms are subjected to submerged conditions and the time intervals for which the microorganisms are subjected to non-submerged conditions have, among themselves respectively, equal or different lengths or are varied in time depending on their number.

8. A device for the microbiological degradation of noxious matters in fluids comprising:
at least two intake spaces (1, 2) for the fluid, immobilized microorganisms being provided in at least one intake space (1, 2);
characterized in that
said at least two intake spaces (1, 2) can be brought in mutual fluid connection through a communication duct (3), and that means (4) are provided by which the fluid can be transferred alternately from one intake space (1) to the other intake space, wherein said microorganisms in said at least one intake space (1, 2) can be alternately subjected to submerged and non-submerged conditions.

9. The device according to claim 8, characterized in that said intake spaces (1, 2) are provided within a container (5) and are separated from each other by at least one dividing wall (6).

10. The device according to claim 8, characterized in that said means (4) comprises a pressing and/or sucking pump for pumping over the fluid between the intake spaces (1, 2).

11. The device according to claim 10, characterized in that said pump (4) changes the hydrostatic pressure prevailing in one of the intake spaces (1, 2) for pumping over the fluid against gravity into the other intake space provided above said one intake space (1), and from there back into said one intake space (1).

12. The device according to any of claims 8 to 11, characterized in that several groups of at least two intake spaces (1, 2) are in fluid connection in parallel and/or in series, and that a means for transferring the fluid from at least one of the intake spaces of one group into at least one of the intake spaces of a neighboring group is provided between said groups of at least two intake spaces.
